(19) United States
(12) Patent Application Publication
(10) Pub. No.: US 2015/0377613 A1
(43) Pub. Date: Dec. 31, 2015

(54) SYSTEMS AND METHODS FOR RECONSTRUCTING 3D SURFACES OF TUBULAR LUMENS

(71) Applicant: Samsung Electronics Co., Ltd., Gyeonggi-do (KR)

(72) Inventors: Eran SMALL, Tel Aviv (IL); Tal Kenig, Avihayil (IL)

(21) Appl. No.: 14/318,744

(22) Filed: Jun. 30, 2014

(51) Int. Cl. G01B 11/25; A61B 1/05; A61B 1/00; A61B 1/303; A61B 1/267; A61B 1/31; A61B 1/273; A61B 1/307; G06T 15/08; A61B 1/06

(52) U.S. Cl. CPC G01B 11/25; G06T 15/08; A61B 1/05; A61B 1/00009; A61B 1/0002; A61B 1/0661; A61B 1/2676; A61B 1/31; A61B 1/2736; A61B 1/307; A61B 1/303; G06T 2210/41

(57) ABSTRACT

There is provided a method for generating a 3D reconstruction of an internal surface of a hollow lumen, comprising: generating a light pattern having a code denoting angular positions; projecting the light pattern onto an internal surface of a tubular lumen; receiving reflections of the light pattern from the internal surface of the tubular lumen; identifying angular positions of the light pattern based on the code; and generating a 3D reconstruction of the internal surface from the received light pattern reflections based on the identified angular positions.

### BACKGROUND

The present invention, in some embodiments thereof, relates to systems and methods for 3D reconstruction and, more specifically, but not exclusively, to systems and methods for 3D reconstruction of inner surfaces of tubular lumens.

The physical world is three-dimensional (3D), yet traditional cameras and imaging sensors are able to acquire only two-dimensional (2D) images that lack depth information. This fundamental restriction greatly limits the ability to measure complex real-world objects.

One principal method of 3D surface imaging is based on the use of structured light. In structured light imaging, the scene is illuminated with a predetermined 2D pattern of parallel lines, or a grid of evenly spaced bars. An imaging sensor is used to acquire a 2D image of the scene illuminated by the structured light. The geometric shape of the scattering surface distorts the projected structured light pattern as seen from the camera. The principle of structured light 3D surface imaging techniques is to extract the 3D surface shape based on the information from the distortion of the projected structured light pattern. The 3D information is extracted using triangulation between the measured position on the camera and the known projected pattern. A calibrated camera maps each of its pixels to a specific 3D vector which represents the light ray collected by the pixel. Using the additional knowledge about the projected pattern, the intersection of this vector and the camera is calculated. This intersection yields the triangulated 3D information.

### SUMMARY

According to an aspect of some embodiments of the present invention there is provided a method for generating a 3D reconstruction of an internal surface of a hollow lumen, comprising: generating a light pattern having a code denoting angular positions; projecting the light pattern onto an internal surface of a tubular lumen; receiving reflections of the light pattern from the internal surface of the tubular lumen; identifying angular positions of the light pattern based on the code; and generating a 3D reconstruction of the internal surface from the received light pattern reflections based on the identified angular positions.

Optionally, the code denotes one or both of a position relative to an optical axis and an arc length.

Optionally, the light pattern has rotational symmetry.

Optionally, projecting comprises projecting the light pattern as a series of coaxial cones having different diverging angles, wherein a circumference of each coaxial cone is coded with the code denoting angular position.

Optionally, the code denoting angular position is selected to increase measurement precision at a direction perpendicular to a vector between a camera receiving reflections of the light pattern and a projector projecting the light patterns.

Optionally, the method further comprises filtering the received light pattern reflection to resolve different parts of the projected light pattern. Optionally, filtering comprises filtering to enhance pseudo-ellipsoidal rings within the received reflected light pattern. Alternatively or additionally, filtering comprises filtering to suppress enhancement of elongated patterns perpendicular to the direction of projected rings of the light pattern. Optionally, filtering is based on the equation:

$${\hat{I}}_{c} ( x ; ξ ) = \{ \begin{matrix}0 , & if {λ}_{2} ( x ) > 0 \\ exp ( - \frac{{R}^{2} ( x )}{2 {σ}_{R}^{2}} ) ( 1 - exp ( - \frac{{S ( x )}^{2}}{2 {σ}_{S}^{2}} ) ) exp ( - \frac{{〈 {v}_{1} ( x ) , D ( x ) 〉}^{2}}{2 {σ}_{D}^{2}} ) , & otherwise\end{matrix}$$

wherein: Î_{c} denotes a filtered image; x denotes a spatial coordinate; ξ denotes the Gaussian derivatives scale; λ₁(x) and λ₂ (x) denote the Hessian eigenvalues at location x,

$$\vert {λ}_{2} ( x ) \vert \geq \vert {λ}_{1} ( x ) \vert ; R ( x ) = \frac{{λ}_{1} ( x )}{{λ}_{2} ( x )} ,$$

the corresponding term promoting elongated structures; S(x)=\sqrt{λ₁²+λ₂²}, is the Frobenius norm of the Hessian matrix, the corresponding term suppressing noise; σ_{R}, σ_{S} are parameters controlling the filtering process; v₁(x) is the Hessian eigenvector corresponding to λ₁, the eigenvalue with the lower magnitude;

$$D ( x ) = \frac{x - {x}_{0}}{‖ x - {x}_{0} ‖} ,$$

is the unit vector pointing from a projected cones axis x₀ to the pixel location x; and σ_{D} is a parameter controlling a directional term.

Optionally, the method further comprises: modulating between projecting the light pattern and projecting multicolored light; receiving reflections of the multicolored light; and registering data based on the received reflection of the light pattern with data based on received reflection of the multicolored light to color the generated 3D reconstruction.

Optionally, the tubular lumen is selected from the group consisting of: trachea, bronchi, colon, esophagus, stomach, duodenum, bladder, fallopian tubes, uterus.

Optionally, the method further comprises repeating the method to sequentially generate multiple 3D reconstructions, and registering the multiple reconstructions to generate a continuous 3D model of the internal surface.

According to an aspect of some embodiments of the present invention there is provided a system for generating a 3D reconstruction of an internal surface of a hollow lumen, comprising:

a source of light for generating a light pattern having a code denoting angular positions, the source of light set for projection of the light pattern onto an internal surface of a tubular lumen;

a sensor for receiving reflections of the light pattern from the internal surface of the tubular lumen;

a processor in electrical communication with the sensor; and

a memory in electrical communication with the processor, the memory having stored thereon:

a module for identifying angular positions of the light pattern based on the code;

and a module for generating a 3D reconstruction of the internal surface from the received light pattern reflections based on the identified angular positions.

Optionally, the system further comprises an endoscope sized for insertion into the tubular lumen, the sensor sized for insertion into the tubular lumen when coupled to a distal end region of the endoscope.

Optionally, the system further comprises expanding optics in optical communication with a diffractive optical element of the source of light, the expanding optics arranged to project the light pattern across a wide field of view including the internal surface of the tubular lumen.

Optionally, the system further comprises: a colored illuminator for projecting colored light on the internal surface; a module for modulating between projection of the light pattern and projection of the colored light; and a module for registering received colored light with received reflection of the light pattern, and for coloring the generated 3D reconstruction based on the registration.

Optionally, the system further comprises a curved mirror positioned distally to the source of light, the mirror sized for allowing some of the projected light pattern to fall on the internal surface distal to the sensor and for reflecting other portions of the projected light pattern to fall on the internal surface proximal to the sensor, the mirror designed based on the projected light pattern to maintain the integrity of the coding during simultaneous proximal and distal imaging. Optionally, the mirror is configured to reflect the light reflected off the internal surface to the sensor. Alternatively or additionally, the mirror is configured such that both the projected light pattern and the light reflected back from the internal surface substantially maintain their respective integrity of the coding when reflected off the mirror.

According to an aspect of some embodiments of the present invention there is provided a method for generating a 3D reconstruction of an internal surface of a hollow lumen, comprising: receiving reflections from the internal surface of a tubular lumen, of a light pattern of projected cones having a code denoting angular positions; filtering the received reflection to suppress enhancement of elongated patterns perpendicular to the direction of the projected cones; identifying angular positions of the light pattern based on the code; and generating a 3D reconstruction of the internal surface from the filtered light pattern reflections based on the identified angular positions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a prior art structured light apparatus, to help understand some embodiments of the present invention;

FIG. 2A is a flowchart of a method of using coded structured light to reconstruct an internal surface of a tubular lumen, in accordance with some embodiments of the present invention;

FIG. 2B is the flowchart of FIG. 2A, with additional optional features, in accordance with some embodiments of the present invention;

FIG. 3 is a block diagram of a system for generating coded structured light to reconstruct an internal surface of a tubular lumen, in accordance with some embodiments of the present invention;

FIG. 4 is a graphical representation of the intersection of different coaxial cones with a virtual colon, in accordance with some embodiments of the present invention;

FIG. 5 is a schematic illustration of a distal end region of an endoscope for generating 3D reconstructed images using structured light, in accordance with some embodiments of the present invention;

FIG. 6 is a schematic illustration of a structured light projector, in accordance with some embodiments of the present invention;

FIG. 7 are exemplary images of concentric ring structured light patterns with and without angular coding, in accordance with some embodiments of the present invention;

FIG. 8 are computer simulated images of 3D colon reconstructions comparing angular coded and un-coded rings, in accordance with some embodiments of the present invention;

FIG. 9 are images illustrating the effects of a filter on the received structured light pattern, in accordance with some embodiments of the present invention;

FIG. 10 is a schematic illustration of blind spots during endoscopic imaging inside a body lumen, in accordance with some embodiments of the present invention;

FIG. 11 is a schematic illustration of an exemplary endoscope with mirror to image both front and back, in accordance with some embodiments of the present invention;

FIG. 12A is a schematic flowchart depicting registration of a 3D image reconstruction with color, in accordance with some embodiments of the present invention; and

FIG. 12B is another schematic flowchart depicting registration of a 3D image reconstruction with color, in accordance with some embodiments of the present invention.

### DETAILED DESCRIPTION

An aspect of some embodiments of the present invention relates to a light pattern having a code denoting angular positions, for generating a 3D reconstruction of an internal surface of a tubular lumen. Optionally, the angular code denotes the position relative to a vector between a projector projecting the light pattern and a camera receiving reflections of the light pattern from the internal surface. Alternatively or additionally, the angular coding denotes an arc length of a portion of the light pattern. The code improves identification of different portions of the light pattern, and/or improves robustness of the 3D reconstruction to high curvatures of the imaged surface, which may undergo strong distortions during the imaging process. The structured light may be used to image a tubular lumen of a patient as part of a medical procedure, optionally as part of an endoscopic procedure.

The angular coding provides another position variable for reconstruction of images, such as within a spherical coordinate system parameterized by (r, θ, φ), where θ denotes the angle relative to the optical axis of the projector, which may be calculated based on identification of projected cones as described herein in further detail. θ may provide enough data to calculate triangulation for reconstruction of the internal surface. φ denotes the angle of the vector between the projector and camera, which may be calculated based on the angular codes. Measuring φ based on the angular coding improves the accuracy of the triangulation for reconstruction of the internal surface. r denotes the radial distance, for example, from the projector to the internal surface.

As used herein, the term internal surface of the tubular lumen is sometimes interchangeable with the term tubular lumen, and is meant to sometimes also include imaging within the space of the lumen itself, in addition to, or instead of, imaging of the internal lumen wall, for example, imaging of blockages, such as debris, feces, or other materials that reside within the space of the lumen.

Optionally, the code is a pattern of predetermined features. The features may be overlaid on the structured light pattern, and/or may be arranged to form the structured light pattern itself.

Optionally, the light pattern has rotational symmetry, for example, the light pattern includes rings, cones, ellipsoids, stars, or other designs.

Optionally, the light pattern is continuously coded. Each separate structure of the pattern (e.g., each ring) may be continuously coded. In this manner, any portion of the received light pattern may be decoded. Alternatively, some of the structures and/or some portions of the pattern are coded, and others remain uncoded.

Optionally, the code denotes angle information, such as angle positions, for the pattern of light. The coded angle information may be used during the 3D reconstruction, to provide information of the angular part of the cone that is being triangulated as part of the reconstruction. The angular information improves measurement precision during the reconstruction. In this manner, the code may improve the accuracy of the 3D reconstruction of the internal surface, providing anatomical information in areas with potential poor precision. The angular coding allows identification of the degree of distortion of the reflected light by the internal surface, and correction of the distortion to generate a more accurate reconstructed image.

Optionally, the light pattern is designed for imaging the internal surface of the tubular lumen, such as the inner wall and/or features on the inner wall. Optionally, the light pattern is rotationally symmetric. The light pattern may include concentric rings, and/or may be generated by a series of coaxial cones having different diverging angles, ellipsoids, and/or other shapes.

In some embodiments, the received reflection of the pattern light is filtered before being analyzed for 3D reconstruction. The filter is designed to resolve the different projected light patterns which have been received after being reflected off the internal surface wall. The filter is designed to help identify individual distinct light patterns (e.g., circles, rings, ellipsoids), which may be overlapping, contacting one another, and/or otherwise smudged together, due to artifacts or other causes, for example, statistical noise, speckle patterns and/or other imperfections in the system.

Optionally, the filter is designed to suppress enhancement of artifacts that are elongated patterns perpendicular to the direction of the projected rings.

In some embodiments, at least some of the light pattern is reflected in a backwards direction off a curved mirror, and at least some of the light pattern is simultaneously projected forwards. The mirror is designed to maintain the integrity of the coding of the light pattern during the reflection and frontal projection. In this manner, the light pattern may be used to generate 3D reconstructed images of regions located behind and/or in front of the sensor and/or projector. Regions that would otherwise remain unimaged blind spots may be imaged using the mirror and angular coded structured light pattern.

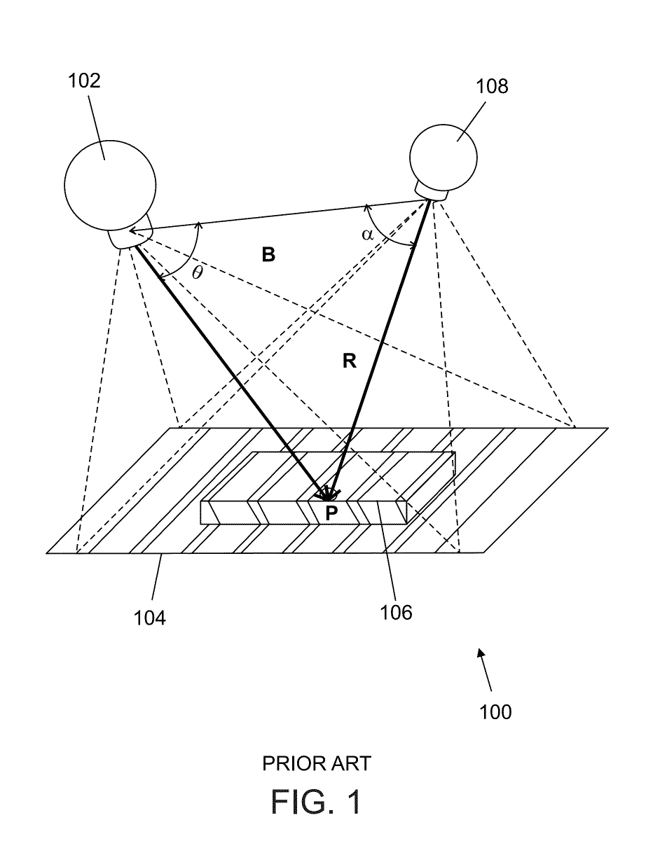

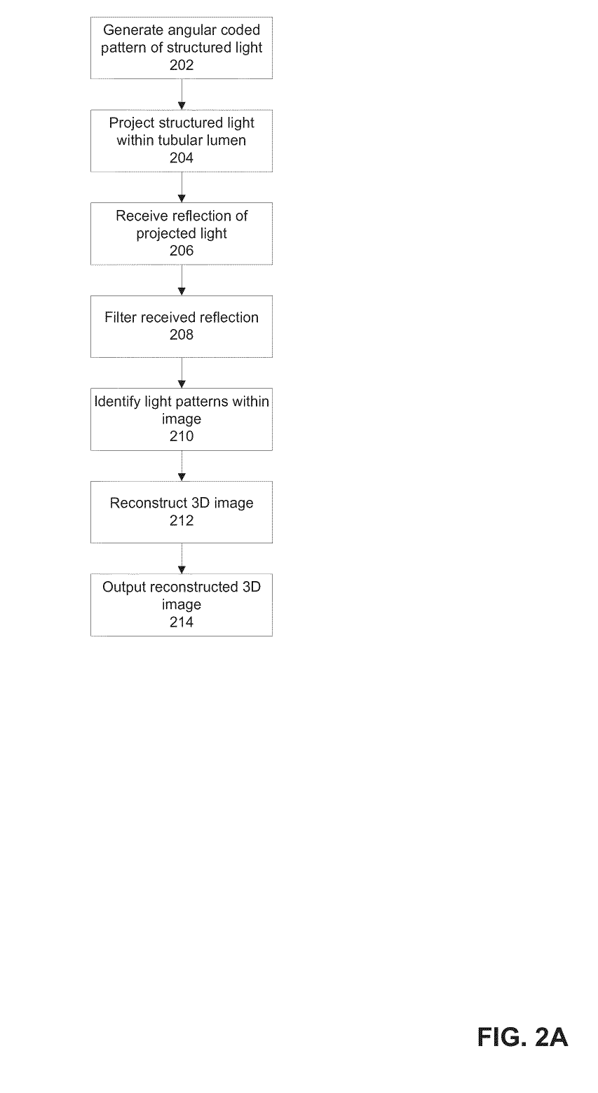

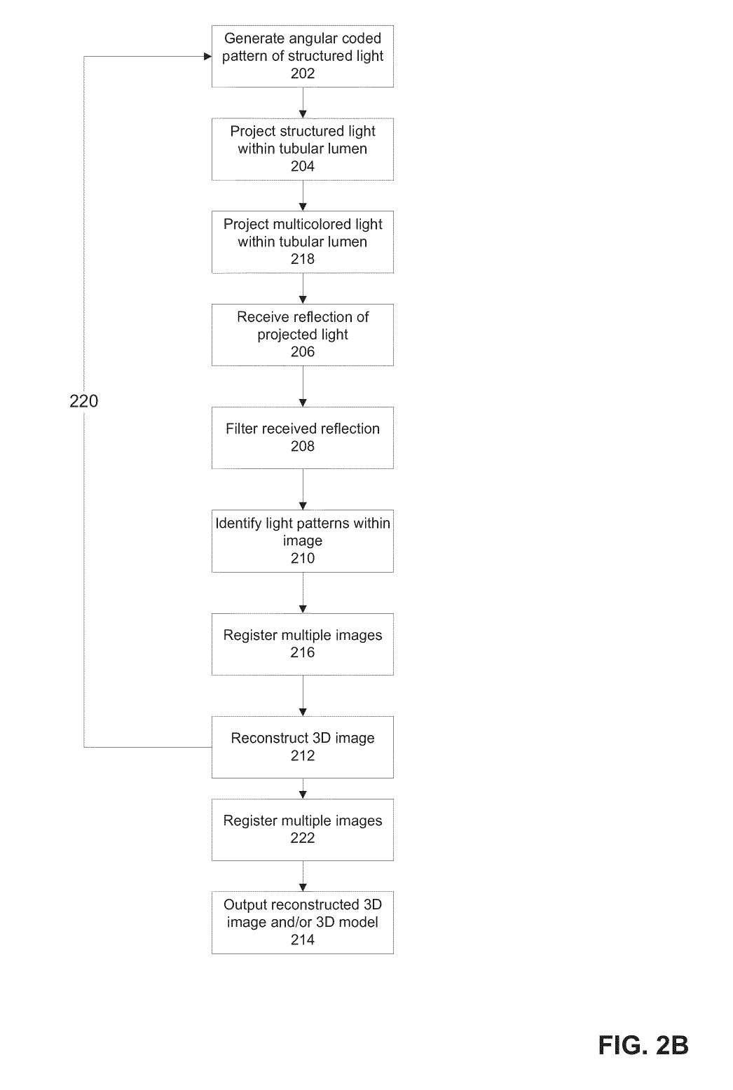

For purposes of better understanding some embodiments of the present invention, as illustrated in FIGS. 2-12 of the drawings, reference is first made to the construction and operation of a structured light apparatus 100 as illustrated in FIG. 1. Structured light apparatus 100 includes a structured light projector 102, such as a laser, for generating a structured light pattern onto a surface. The structured light is designed for projection onto a generally flat surface 104 with a feature 106. The structured light pattern of horizontal bars or a grid, is designed for projection onto flat surface 104. The light reflecting off flat surface 104 with feature 106 is received by a camera 108. A 3D reconstruction of surface 104 and/or feature 106 is generated based on the measured distortion of the sensed reflected light pattern, the known pattern of projected structured light, and/or angular information between the light projection, the camera and/or the surface.

Inventors realized that structured light systems designed for imaging flat surfaces (that may include features) may not be adequate for imaging internal surfaces of tubular lumens. As described herein, inventors designed a structured light system and/or method for improved imaging of the internal surface of a tubular lumen.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a non-transitory computer readable storage medium (or media) having computer readable program instructions thereon for causing a processor to carry out aspects of the present invention. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

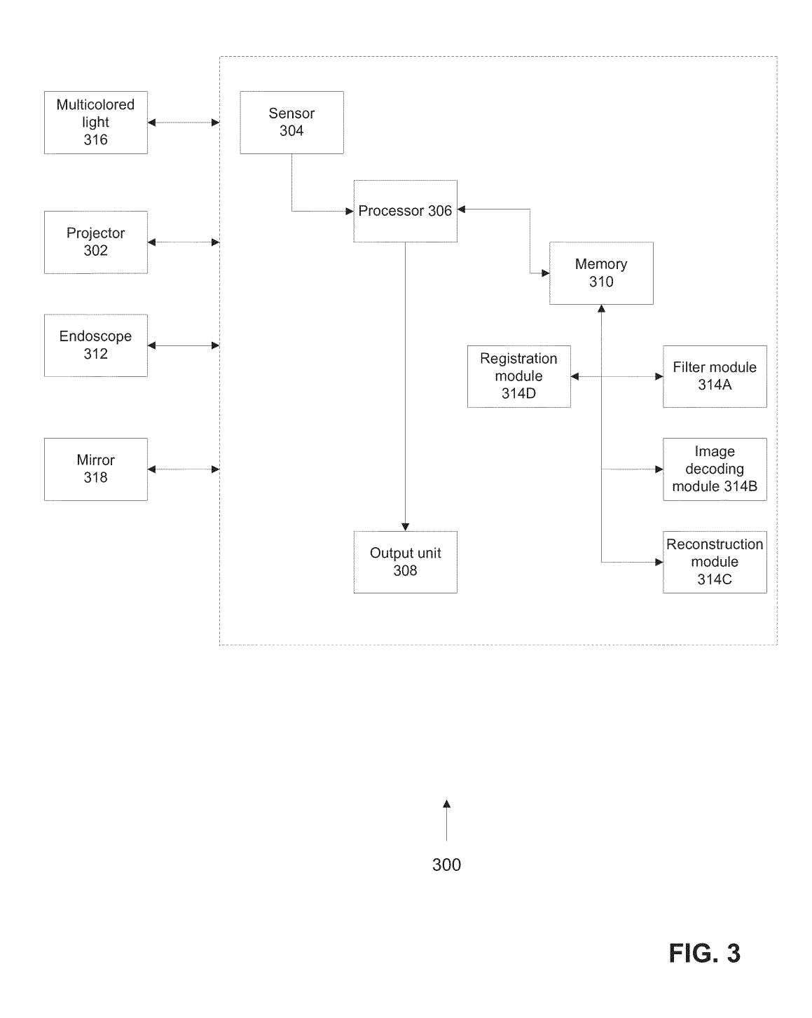

Reference is now made to FIG. 2A, which is a flowchart of a method of generating a 3D structure of an internal wall of a tubular lumen based on angular coded structured light, in accordance with some embodiments of the present invention. Reference will also be made to FIG. 3, which is a block diagram of a system 300 for generating a 3D structure of an internal wall of a tubular lumen based on angular coded structured light, in accordance with some embodiments of the present invention. The method of FIG. 2A may be performed by the system of FIG. 3.

The system and/or method may provide robustness to optical aberrations caused by strong optical elements, such as optical elements used with compact wide Field-of-View (FoV) endoscopic systems. The system and/or method may provide robustness to identification of individual circles (or other patterns) when reflected by highly curved surfaces such as folds inside tubular lumens and/or organs. The system and/or method may provide robustness to identification of individual circles (or other patterns) when the received image has been affected by artifacts such as noise, speckles and/or other optical imperfections. The system and/or method may provide high precision triangulation at all available viewing directions.

System 300 includes a projector 302 for projecting a structured light pattern onto the inner surface of a tubular lumen, and a sensor 304 for sensing the light pattern reflected from the inner surface. The light signals received by sensor 304 are processed by one or more processors 306, which may generate a 3D reconstruction of the inner surface. The 3D reconstruction may be displayed on a screen, stored in a data repository, and/or forwarded, by an output unit 308.

Processor 306 may be in electrical communication with a memory 310 having stored thereon one or more modules having instructions for execution by processor 306.

Optionally, system 300 includes an endoscope 312, for imaging the internal body of a patient. The components of system 300 may be designed for attachment to an existing endoscope, may be part of a custom designed imaging probe, and/or may be integrated within endoscope designs.

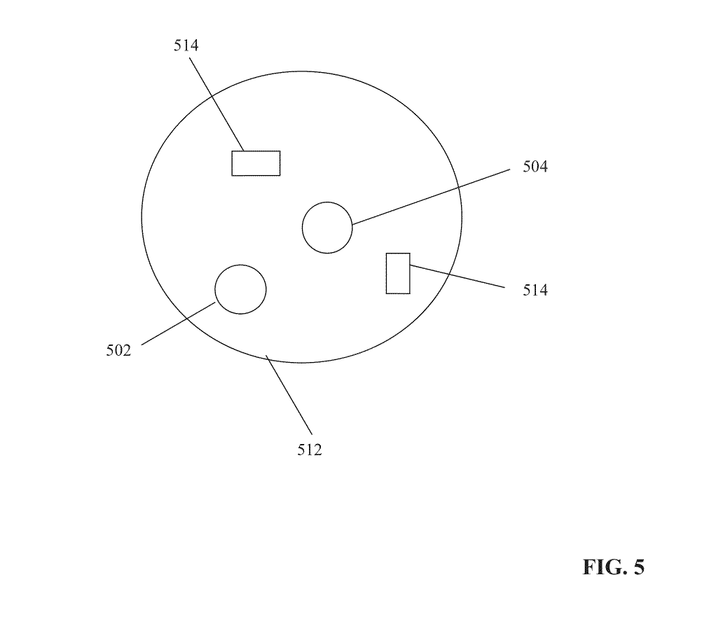

Reference is now made to FIG. 5, which is a schematic illustration of a distal end region of an endoscope 512 for generating 3D reconstructed images using angular coded structured light, in accordance with some embodiments of the present invention. Endoscope 512 is shown face-on. A projector 502 projects angular coded structured light. A sensor 504 senses the reflected structured light. Projector 502 and/or sensor 504 are sized for positioning on the distal end region of endoscope 512, for insertion into body lumens. Optionally, one or more multicolored light sources 514, such as white light generators, such as a light emitting diode (LED) project multicolored light, such as white light. The white light may be used to visually look at the lumen, and/or to obtain color data of the inner surface, which may be registered with the 3D reconstruction to color the 3D reconstruction, as described herein.

The structured light system and/or method described herein may be implemented for use as part of minimally invasive endoscopic medical procedures and/or other, non-medical applications requiring 3D reconstruction of internal tubular structures and/or organs. In this way a 3D model of the interior of an organ and/or tissue may be reconstructed, for example, the interior of the colon, the esophagus, the stomach, the duodenum and/or other parts of the small intestine, the trachea, the bronchi, the bladder, the fallopian tubes, the uterus, and/or other parts of the body. Some parts of the body may be expanded for imaging using saline or other fluids that allow projection and reception of light. In medical applications, the systems and/or methods may provide additional anatomic information that may help to improve diagnosis and/or treatment.

As described herein, endoscopy serves as an exemplary application of the coded structured light system and/or method. However, as may be understood from the descriptions herein, the systems and/or methods are not confined to endoscopy or medical usage only.

Referring back to FIG. 2, at 202, an angular coded pattern of structured light is generated, for projection against the internal surface of the tubular lumen.

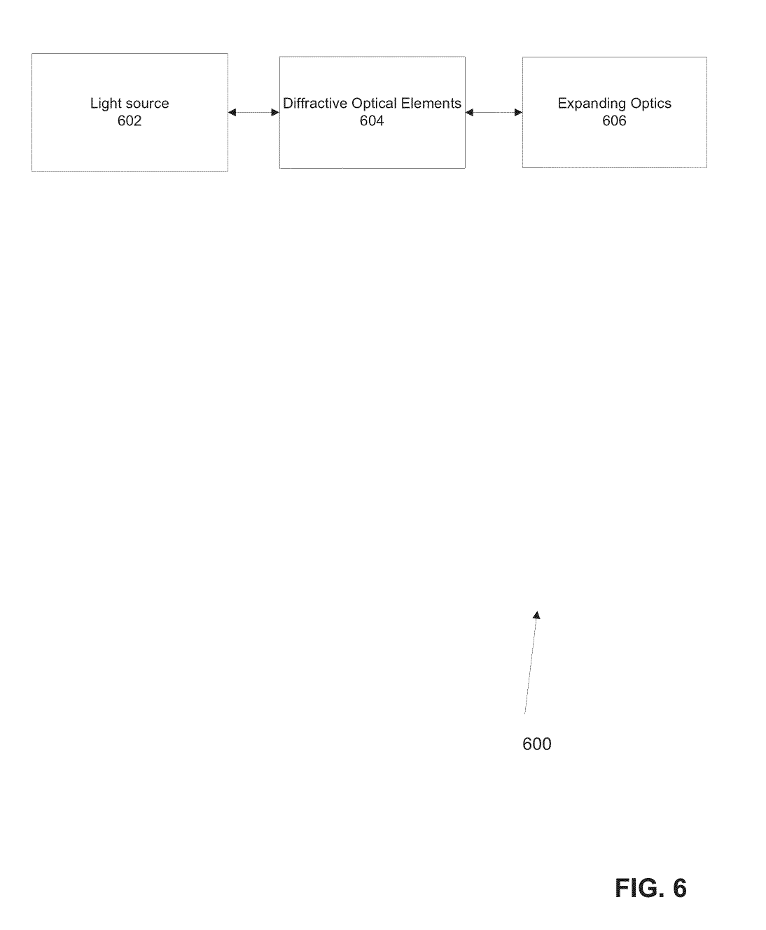

Reference is now made to FIG. 6, which is a schematic illustration of a structured light projector 600, in accordance with some embodiments of the present invention. Projector 600 may be used to generate the angular coded pattern of structured light as in block 202 of FIG. 2A. Projector 600 may be used as projector 302 of system 300 of FIG. 3, and/or projector 502 of endoscope 512 of FIG. 5.

Optionally, projector 600 comprises a light source 602, optionally a monochromatic light source, for example, a laser diode. Light source 602 may reside within projector 600, and/or may reside externally, being delivered to projector 600 by an optical fiber.

Optionally, a diffractive optical element (DOE) 604 is optically coupled to light source 602, to generate the angular coded pattern of structured light as described herein. DOE 604 may be fixed in hardware, repeatedly generating the same pattern of structured light, and/or may be adjustable, such as by software or other circuitry, to adjust the pattern of structured light.

Optionally, expanding optics 606 are optically coupled to DOE 604, to spread out the light pattern for imaging the field of view within the tubular lumen. Optionally, optics 606 diverge the structured light based on the field of view of the sensor receiving the reflected light. Expanding optics 606 may include one or more lenses in optical communication with one another, for example, a plano-convex lens in optical communication with DOE 604, a concave-concave lens in optical communication with the plano-convex lens, and a plano-concave lens in optical communication with the concave-concave lens.

Expanding optics 606 may introduce optical aberrations to the structured light, which may be prevented, reduced and/or corrected by the methods and/or systems described herein.

Referring back to block 202 of FIG. 2A, optionally, the generated light pattern has a continuously coded pattern. Optionally, the generated light pattern has rotational symmetry.

The generated light pattern may be concentric rings, circles, ellipsoids, or other shapes. The rotational symmetry may be around the optical axis.

Optionally, the rotation symmetry and/or shape of the generated light pattern is selected based on the expected pattern of the internal surface of the lumen. Selection of the light pattern having circular symmetry based on an approximately circular internal surface may be robust to optical aberrations. The optical aberrations may be due to, for example, strong optical elements that introduce the optical aberrations. The strong optical elements may be part of a small system for imaging body lumens (e.g., endoscope), the elements designed to obtain large divergence angles of the generated light pattern in order to achieve very wide FoV, to image the sides of the internal surface of the tubular structure. In order to 3D reconstruct the entire camera's view, the structured light pattern may be passed through the optical elements to obtain the divergence angles similar to the FoV of the camera.

The tubular lumen may be located in a patient. The tubular lumen may contain curved and/or folded surfaces, for example, the semilunar folds of the colon, the longitudinal folds of the trachea, and/or other features. Internal organs imaged for 3D reconstruction may consist of curved features having large variations. When images are acquired from very close distances inside the organ, the imaged structured light pattern may undergo strong distortions. The selected pattern of predetermined continuous features may ease the process of identifying the individual light patterns, and/or may improve robustness to curvatures of the imaged object.

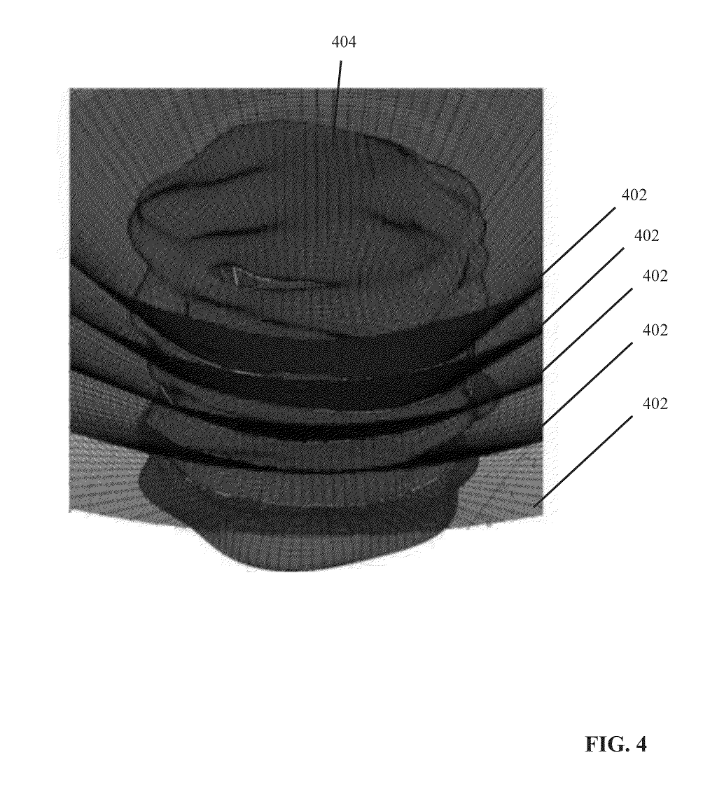

Optionally, the light is generated as a pattern of a series of coaxial cones, such as by projecting the light in different coaxial cones with different diverging angles. Inventors discovered that the pattern of coaxial cones may improve 3D reconstruction images for tubular geometries. FIG. 4 is a graphical representation simulating the intersection of different coaxial cones 402 with a virtual colon 404, in accordance with some embodiments of the present invention. Inventors discovered that the coaxial cones pattern may remain coaxial under optical aberrations, and/or may yield an easily identifiable image made out of continuous rings.

Optionally, the light pattern is coded with a code denoting angular information. Each light pattern (e.g., circle) may be coded with the same code, or different light patterns may be coded with different codes.

Optionally, angular information is derived based on angular coding of each (or some) cone. The coding may be one-dimensional, or two-dimensional. Optionally, the circumference of each (or some) cone is coded. The coding enables estimation of the angular part of the cone being inspected.

The coding may be selected based on a desired tolerance range for the error in angle measurement. Optionally, the coding is selected to limit the angular uncertainty at the detected area to a small angular range. The smaller the angular range, the more isotropic the triangulation algorithm's precision for 3D reconstruction. Optionally, the coding is selected based on a tradeoff between improved isotropic 3D reconstruction and smaller angular uncertainty.

The coding may denote an angle certainty of and/or an arc length of, for example, about 36 degrees, or about 18 degrees, or about 15 degrees, or about 10 degrees, or about 9 degrees, or about 4.5 degrees, or about 2 degrees, or about 1 degree, or other smaller intermediate or larger angle ranges.

Coding may include, for example, alternating bands of light and dark, where each light and/or dark band represents a predetermined angle range, modulation of the line itself, such as changes in thickness, modulation of a frequency related pattern, such as a sinusoidal pattern in the shape of a circle, or other coding methods.

Optionally, the code denoting angular positions is selected to increase measurement precision at a direction perpendicular to a vector between a sensor receiving reflections of the light pattern (e.g., sensor 304) and a projector projecting the light patterns (e.g., projector 302).

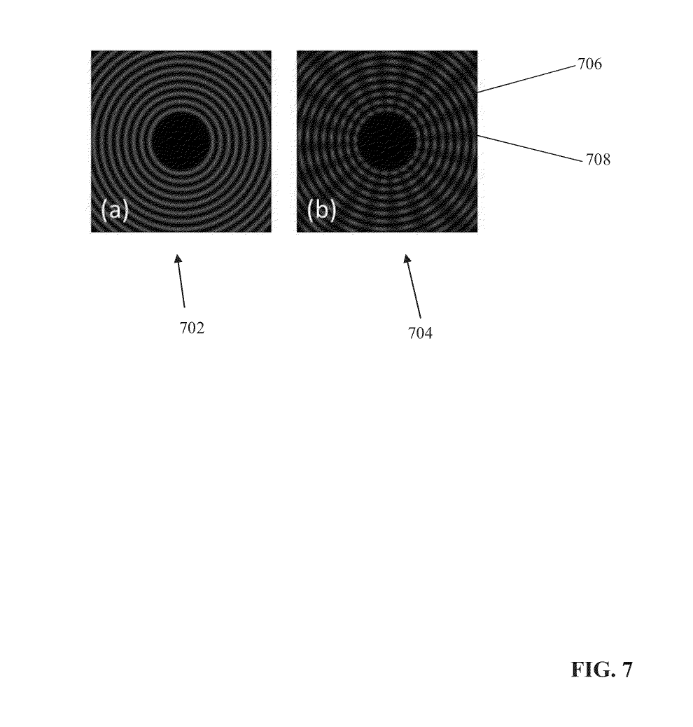

Reference is now made to FIG. 7, which includes exemplary images of concentric ring structured light patterns with and without angular coding, in accordance with some embodiments of the present invention. It is noted that the coded patterns depicted in FIG. 7 are not necessarily limiting, as other possible coded patterns may be used.

The image on the left (labeled 702) is a concentric ring pattern without angular coding. With reference to the (r, θ, φ) spherical coordinate system, θ may be calculated from the ring pattern of image 702. The image of the right (labeled 704) is a concentric ring pattern with angular coding. φ may be calculated based on the angular coding of image 704. The angular coding comprises a pattern of alternating light 706 and dark 708 bands. Each light and/or dark band denotes a certain angular range. The light and/or dark bands may be evenly spaced and/or evenly sized, denoting similar angle ranges (as shown). The light and/or dark bands may be differently spaced and/or differently sized, denoting different angle ranges. In this manner, distortions of the ring pattern may be identified, by calculating the angle ranges based on the received light and/or dark patterns.

Referring back to FIG. 2A, optionally at 204, the structured light pattern is projected onto an internal surface of a tubular lumen. The structured light pattern may be projected, for example, using projector 302 of FIG. 3.

Optionally, at 206, the reflections of the projected light pattern from the internal surface of the tubular lumen are received. The reflections may be received, for example, by sensor 304 of FIG. 3.

Optionally, at 208, the reflected image and/or data is processed, such as by a filter. The filter may be executed in hardware and/or in software, for example, by a filter module 314A.

The filter is designed to filter the image to help resolve different projected light patterns. Speckle patterns, statistical noise and/or other imperfections of the endoscopic system may cause parts of the reflected concentric ring patterns to appear blended together, and/or to appear overlapping and/or continuous with each other. The ring blending may occur when imaging non-tubular structures, such as planar surfaces, for example, during a projector calibration process.

The filter may incorporate known geometric properties of the projected pattern. The filter may be designed to enhance elongated structures within the image, such as the projected cone patterns, which may be pre-defined be composed of pseudo-ellipsoidal rings, which are elongated structures. Optionally, the filter is designed to enhance pseudo-ellipsoidal rings within the received reflected light pattern. Alternatively or additionally, the filter is designed to suppress enhancement of elongated patterns perpendicular to the direction of projected rings of the light pattern.

An exemplary implementation of the filter is now described. The exemplary filter is designed based on the pre-defined ellipsoidal shape of the projected pattern for guiding the filtering scheme. The exemplary filter may be represented by Equation 1:

$${\hat{I}}_{c} ( x ; ξ ) = \{ \begin{matrix}0 , & if {λ}_{2} ( x ) > 0 \\ exp ( - \frac{{R}^{2} ( x )}{2 {σ}_{R}^{2}} ) ( 1 - exp ( - \frac{{S ( x )}^{2}}{2 {σ}_{S}^{2}} ) ) exp ( - \frac{{〈 {v}_{1} ( x ) , D ( x ) 〉}^{2}}{2 {σ}_{D}^{2}} ) , & otherwise\end{matrix}$$

### Where:

Î_{c} denotes a filtered (e.g., circle enhanced) image;
x denotes a spatial coordinate (e.g., pixel location);
ξ denotes the Gaussian derivatives scale;
λ₁(x) and λ₂(x) denote the Hessian eigenvalues at location x, |λ₂(x)|≧|λ₁(x)|;

$$R ( x ) = \frac{{λ}_{1} ( x )}{{λ}_{2} ( x )} ,$$

the corresponding term promoting elongated structures;
S(x)=\sqrt{λ₁²+λ₂²}, is the Frobenius norm of the Hessian matrix, the corresponding term suppressing noise;
σ_{R}, σ_{S} are parameters controlling the filtering process;
v₁(x) is the Hessian eigenvector corresponding to λ₁, the eigenvalue with the lower magnitude;

$$D ( x ) = \frac{x - {x}_{0}}{‖ x - {x}_{0} ‖} ,$$

is the unit vector pointing from the projected cones axis x₀ to the pixel location x; and
σ_{D} is a parameter controlling a directional term.

The 2×2 Hessian matrix (second order spatial derivatives) may be calculated per pixel location within the image. In order for the filter to provide strong response for specific scales, Gaussian derivatives of a certain scale may be used.

Eigenvalue decomposition is performed on the Hessian matrix, yielding two eigenvectors and corresponding eigenvalues. The eigenvectors provide two perpendicular directions, corresponding to the directions of maximal and minimal curvatures. The eigenvalues indicate the curvature magnitude along the eigenvector directions.

In order to fuse data from multiple scales, the maximal filter response is taken for each pixel location using Equation 2:

$${I}_{c} ( x ) = {max}_{ξ} {\hat{I}}_{c} ( x ; ξ )$$

Where ξ varies within a predetermined range, designed to cover the range of all scales within the image.

X₀, which denotes the location of the projected cones axis, may be detected by locating the laser zero order diffraction. The zero order diffraction produces a spot of high intensity around the projected axis, and may be detected, for example, by thresholding the image and calculating the center of mass of the high intensity region.

D(x) is expected to be oriented perpendicular to the circular pattern. Therefore, when v₁(x) is directed along the circular pattern, the inner product vanishes, which causes the associated term in Eq. 1 to be maximal. On the other hand, at locations where v₁(x) is oriented perpendicular to the circular pattern, the inner product [symbol] v₁(x),D(x) [symbol] approaches its maximal value of 1, and the associated term is suppressed.

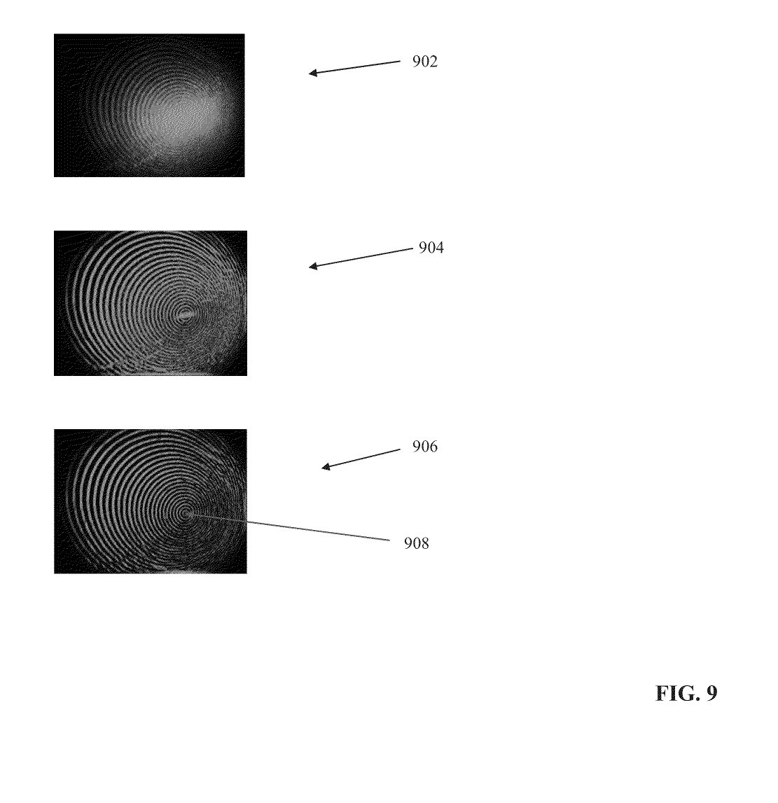

Reference is now made to FIG. 9, which are images illustrating the effects of the filter on the received structured light pattern, in accordance with some embodiments of the present invention. An exemplary received image with artifacts is compared, using the exemplary filter described herein (e.g., Equations 1 and 2) with another filter.

Image 902 denotes an acquired projected pattern image, having artifacts, for example, as a result of noise, speckle, and/or other imperfections in the imaging system. Individual rings appear blended and/or continuous with one another in certain regions of the image. The individual rings may be difficult to separate from one another to distinctly identify in the blended and/or continuous regions.

Image 904 denotes the acquired image after being processed by another filter, such as a vessel enhancement filter as described with reference to “Frangi, A. F., Niessen, W. J., Vincken, K. L., & Viergever, M. A. (1998). Multiscale vessel enhancement filtering. In Medical Image Computing and Computer-Assisted Intervention—MICCAI'98 (pp. 130-137). Springer Berlin Heidelberg”, incorporated herein in its entirety.

Image 906 denotes the acquired image after being processed by the exemplary filter (i.e., Equations 1 and 2) described herein. The projected cones axis X₀ is denoted by arrow 908.

Referring back to FIG. 2A, at 210, the image is decoded to acquire the structured light pattern. The individual projected cones are identified from the acquired image. Points in the image domain are localized and attributed to specific cones. The image may be decoded, for example, by an image decoding module 314B.

Optionally, angular positions of the light pattern are identified based on the angular coding.

Optionally, at 212, a 3D reconstruction of the internal surface is generated from the received light pattern reflections, based on the identified angular positions. The 3D reconstruction may be performed, for example, by a 3D reconstruction module 314C. Alternatively or additionally, the 3D reconstruction may be performed by a different system and/or at a different location. For example, the filtered image, optionally with the identified light cones, may be forwarded over a network to a remote server for generating the 3D reconstruction.

The angle coding may provide isotropic precision of the triangulation process along the projected circle and/or cone. Each of the rings (or circles) sensed by the sensor has regions which may be subject to more precise triangulation than others. The angular information may even out the precision between regions, to generate isotropic precision. The 3D reconstruction may be based on triangulation of the intersection between a vector and an identified cone. The degree of accuracy achievable by the triangulation process may depend on the relative direction between the projector and the camera. The angular coding may prevent areas with very poor precision where anatomical information may be lost.

The angular coding along the projected circle and/or code may improve angular certainty in the detected circle and/or cone. The triangulation for 3D reconstruction between a vector and the cone may be provided with information regarding what angular part of the cone is being triangulated. The angular information may improve measurement precision at the direction perpendicular to the vector between the sensor sensing the reflected light and the projector generating the light pattern.

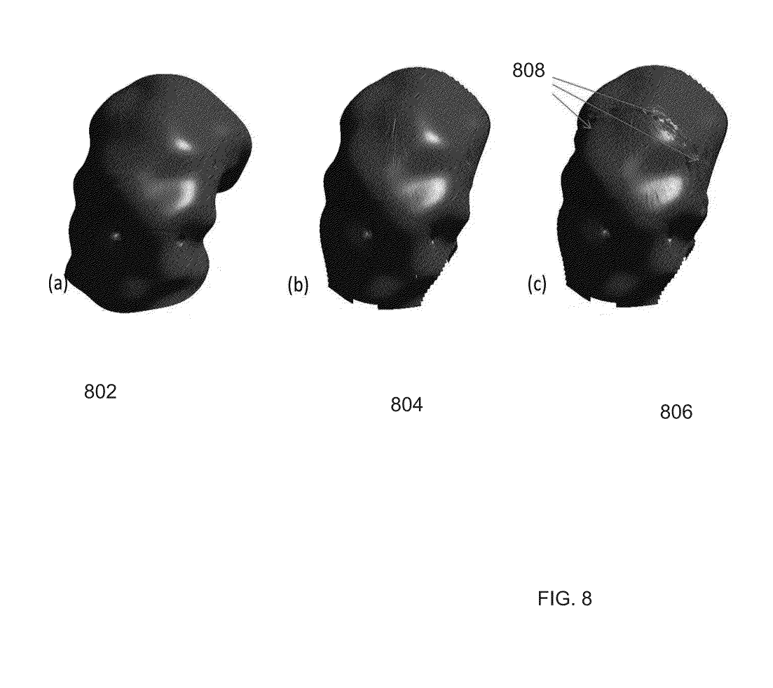

Inventors performed experiments, using computer simulations, to illustrate the effects of angular coded light pattern rings in comparison to un-coded rings. Reference is now made to FIG. 8, which is computer simulated images of 3D colon reconstruction comparing angular coded and un-coded ring light patterns, in accordance with some embodiments of the present invention. FIG. 8 illustrates an exemplary improvement in 3D reconstruction using angular coded rings in comparison to un-coded rings.

The figure on the left (labeled 802) denotes a virtual computer generated colon model, to be reconstructed based on structured light rings. The figure in the middle (labeled 804) denotes a reconstruction of the colon based on coded rings, as described herein. The figure on the right (labeled 806) denotes a reconstruction of the colon based on un-coded rings. Regions denoted by arrows 808, where the reconstructed surface intersects the YZ plane (X=T0), have stronger fluctuations compared to the rest of the reconstructed surface. The regions denoted by arrows 808 represent low precision areas. Corresponding regions in reconstructed structure 806 do not have such low precision areas. Reconstruction 806 exemplifies the use of the coded pattern in obtaining a reconstruction with more uniform precision. Angular coding of structured light pattern comprised of cones may improve precision of the 3D reconstruction.

Referring back to FIG. 2A, optionally at 214, the reconstructed 3D image is outputted, for example, by output unit 308. The reconstructed 3D image may be displayed, such as on a screen, may be saved, such as within a data repository on a memory, and/or may be forwarded, such as using a network to a remote processor.

Reference is now made to FIG. 2B, which is the flowchart of FIG. 2A with additional optional features, in accordance with some embodiments of the present invention. The additional optional features of the method may be performed, for example, by system 300 of FIG. 3 and/or other systems described herein. For brevity, the additional blocks and/or modification to existing blocks are described.

As used herein, the term proximal means closer to the operator of an endoscope, or in a direction towards the outside of the patient when the endoscope is located inside the patient. As used herein, the term distal means further away from the operator, or in a direction deeper inside the patient when the endoscope is inside the patient.

Referring back to FIG. 3, optionally system 300 includes a mirror 318 positioned distally to the structured light projector, the mirror sized for allowing some of the projected structured light pattern to fall on the internal surface distal to the sensor and for reflecting other portions of the projected light pattern to fall on the internal surface proximal to the sensor, the mirror designed based on the projected light pattern to maintain the integrity of the angular coding. The mirror may enable detection of tumors and/or lesions that may not be detected using conventional endoscopic devices. Optionally, the mirror is configured to reflect the light reflected off the internal surface to the sensor. The mirror is configured (e.g., designed and/or positioned) such that both the projected light pattern and the light reflected back from the internal surface (or other imaged object) substantially maintain their respective integrity of the coding when reflected off the mirror. The integrity of the coding may be maintained to enable 3D reconstruction with the same level of accuracy as would be achieved without the mirror affecting the integrity of the coding. Some reduction in integrity by the mirror may be allowed, for example, defined by an integrity threshold.

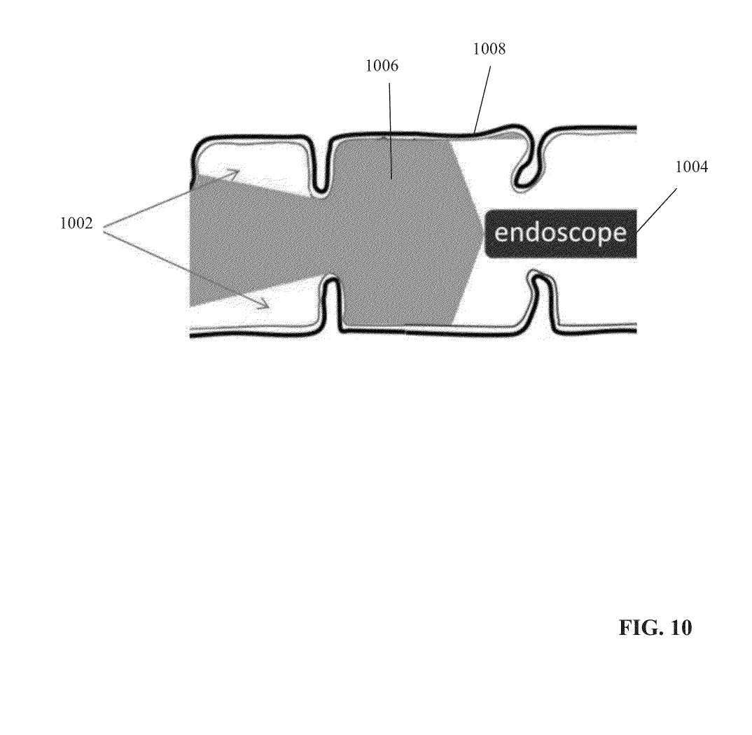

Reference is now made to FIG. 10, which is a schematic illustration of blind spots 1002 during imaging with an endoscope 1004 having a light source projecting structured light 1006 inside a body lumen 1008, in accordance with some embodiments of the present invention. Mirror 318 may help imaging of blind spots. Such blind spots may be formed when the FoV of the sensor is less than 180 degrees, so that the entire distal front may not be imaged at the same time. Such blind spots may be formed when the internal lumen contains internal folds that may block certain portions of the surface from being imaged. The blind spots may lead to partial loss of information when imaging, for example, increasing the miss rate of tumors.

Referring back to FIG. 3, mirror 318 allows for simultaneous imaging of both fields distal to the sensor and/or projector, and proximal to the sensor and/or projector. The mirror may be curved and/or axicon. The mirror may be designed to maintain the integrity of the angular coding, for example, the mirror is continuous and/or has rotational symmetry corresponding to the angular coding. Light reflected off the mirror may maintain the integrity of the angular code. The received light may be analyzed based on the maintained integrity of the angular code.

Optionally, mirror 318 is set to be positioned along the longitudinal axis of the endoscope. In this manner, mirror 318 allows for distal imaging behind the sensor, at a trade-off of reduced proximal imaging of approximately the center of the lumen. The imaging of the center of the lumen may be traded away in this manner, as the side views of the camera may improve visualization with fewer blind spots. The front view may be utilized for overcoming the blind spots and/or adding the information to the reconstruction.

Optionally, at 216, multiple images obtained by the sensor with positioned mirror 318 are registered, for example, by a registration module 314D. The multiple registered images may be integrated together to obtain a single 3D reconstruction of the internal lumen (e.g., block 212).

A more complete model of the internal luminal wall (e.g., of the organ) may be reconstructed by scanning the interior of the lumen with the endoscope, registering and/or integrating multiple surfaces into a single model. Registration of different reconstructed surfaces during the course of the scan may provide for a model of the object or organ to be reconstructed using only (or mostly) the side view images.

The rearview image may have different blind spots (e.g., on the other side of internal lumen folds) from the front-view, which may achieve a view point of the same structure from opposite angles. These two views may be registered to reconstruct the entire surface feature without any (or with reduced) blind spots.

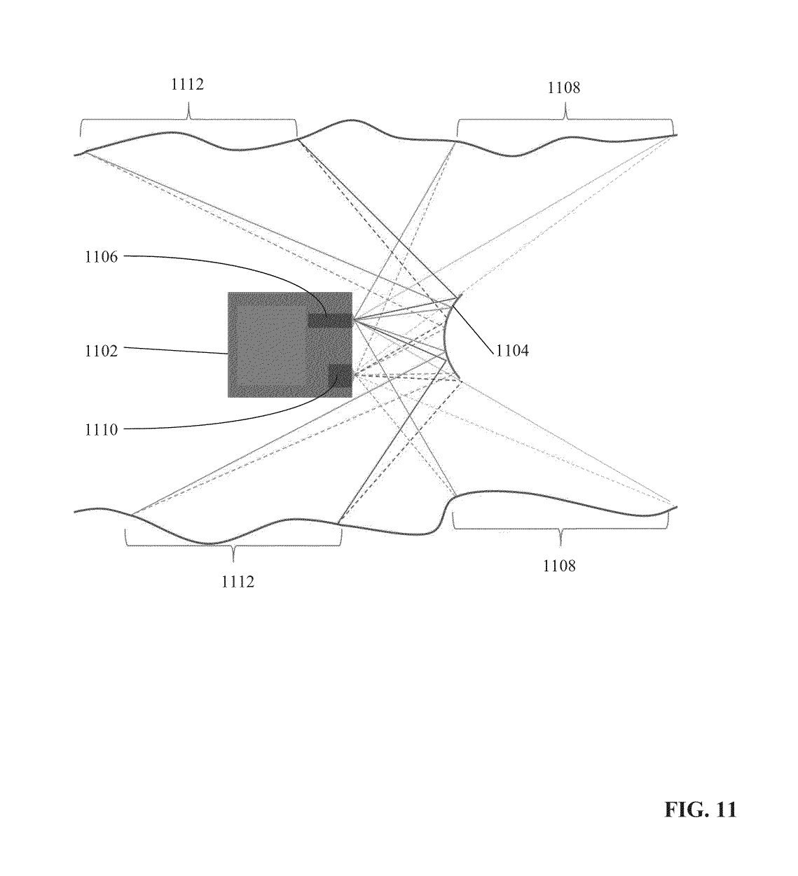

Reference is now made to FIG. 11, which is a schematic illustration of an exemplary endoscope 1102 with mirror 1104 to image both front and back (i.e., distally and proximally), in accordance with some embodiments of the present invention. The system of FIG. 11 may provide simultaneous front and rear views, may provide data for 3D reconstruction of front and rear views based on angular coded structured light, and/or may prevent or reduce blind spots based on multi-view registration.

Mirror 1104 is positioned approximately in the center of view of endoscope 1102, as described herein. Mirror 1104 may be positioned along the longitudinal axis of endoscope 1102, the reflective surface of mirror 1104 approximately perpendicular to the longitudinal axis. Endoscope 1102 may be used to perform the method of FIG. 2B.

At least some structured light with angular coding (denoted by solid lines) projected from projector 1106 falls distally of the distal end region of endoscope 1102, on front view regions 1108 located on the inner wall of a lumen. The light is reflected back (denoted by dotted lines) and sensed by sensor 1110. In this manner, data of the side views of the inner wall of lumen distal to endoscope 1102 is gathered.

At least some structured light with angular coding (denoted by solid lines) is reflected by mirror 1104, and falls proximally of the distal end region of endoscope 1102, on rear view regions 1112 located on the inner wall of the lumen. The light is reflected back (denoted by dotted lines) and sensed by sensor 1110. In this manner, data of the side view of the inner wall of lumen proximal to the distal end region of endoscope 1102 is gathered.

The structured light may be projected to the front and back, for example, in block 204. The light reflected from the front and back may be received, for example, in block 206.

The arrangement depicted in FIG. 11 enables sensor 1110 to obtain data for generating 3D reconstructions of both front and rear views. The 3D reconstruction may be performed, for example, in block 212.

Alternatively or additionally, mirror 1104 may be used for visualization, and/or for adding texture and/or object color using multicolored light (e.g., white), for example, as described herein. The structured light (which may be monochromatic) and the multicolored light may be alternated, to reconstruct 3D structures having true color and/or texture of the images surface features and/or objects, for example, as described herein.

Referring back to FIG. 3, system 300 optionally includes a multicolored light source 316, for projecting multicolored light, for example, white light. Multicolored light source 316 generates light of different colors, the reflection of which provides data for registering with the 3D reconstructed images to add color to the 3D images. Optionally, high color resolution data is added to the 3D reconstruction. The color may enable better differentiation between close color shades. The color may improve diagnostic precision, for example, better tumor identification, and/or identification of other abnormalities within the lumen. The color registration may improve the distinction of structures and/or textures in general imaging, and/or in specific applications such as in endoscopy. The detection rate of pre-cancerous and/or cancerous tumors inside the lumen may be increased when the reconstructed images are enhanced with color provided by multicolored light source 316.

Optionally, light source 316 and/or projector 302 are located externally to the probe (e.g., endoscope) inserted within the lumen. Light source 316 and/or projector 302 may be optically coupled to the distal end region of the probe, such as by an optical fiber. In this manner, sources of light that would not fit into the lumen, such as multi-colored light sources 316 generating light of multiple wavelengths, for example a white light source, and/or projector 302 which may generate light of a single wavelength, may be used to direct light within the lumen.

Alternatively or additionally, multicolored light source 316 and/or projector 302 are designed to fit on endoscope 312, and sized for insertion into the lumen, for projecting light within the lumen.

Reference is now made back to FIG. 2B, to describe an exemplary method of adding color to 3D images, in accordance with some embodiments of the present invention.

Optionally, at 218, multicolored light such as white light is projected into the lumen, for example, using a white light emitting diode (LED) from multicolored light source 316 of FIG. 3, or other sources.

Optionally, projection of the structured light (e.g., block 204) and projection of the multicolored light are modulated, between one another. The projections may be repeatedly alternated. The structured light may be monochromatic, such as laser light.

Optionally, the modulation acquires both 3D information regarding the lumen (as described herein), and color information regarding the lumen.

Optionally, at 216, the color data is registered with the 3D information. The 3D data may be colored based on the registered color data.

At 212, a colored 3D reconstruction is generated.

Optionally, at 220, the method of FIG. 2B is repeated to generate multiple reconstructed 3D images. The multiple images may be sequential images, such as obtained sequentially. The multiple images may be of different parts of the lumen, for example, images including the entire clinically significant length of the lumen, such as obtained by advancing the endoscope within the lumen. The multiple images may be of different portions within the lumen, for example, portions of the internal wall, such as acquired by rotating the endoscopic head within the lumen, and/or acquiring front and/or back facing views (for example, as described herein).

The generated multiple reconstructed 3D images may be of a single color, and/or colored as described herein.

Optionally, at 222, the multiple reconstructed 3D images are registered, to generate a reconstructed 3D model of the lumen. The 3D model may be continuous. For example, multiple images of the colon are registered together to obtain a complete 3D model of the internal surface of the colon.

Optionally, at 214, the reconstructed 3D model of the lumen is outputted, as described herein in reference to outputting of the 3D image.

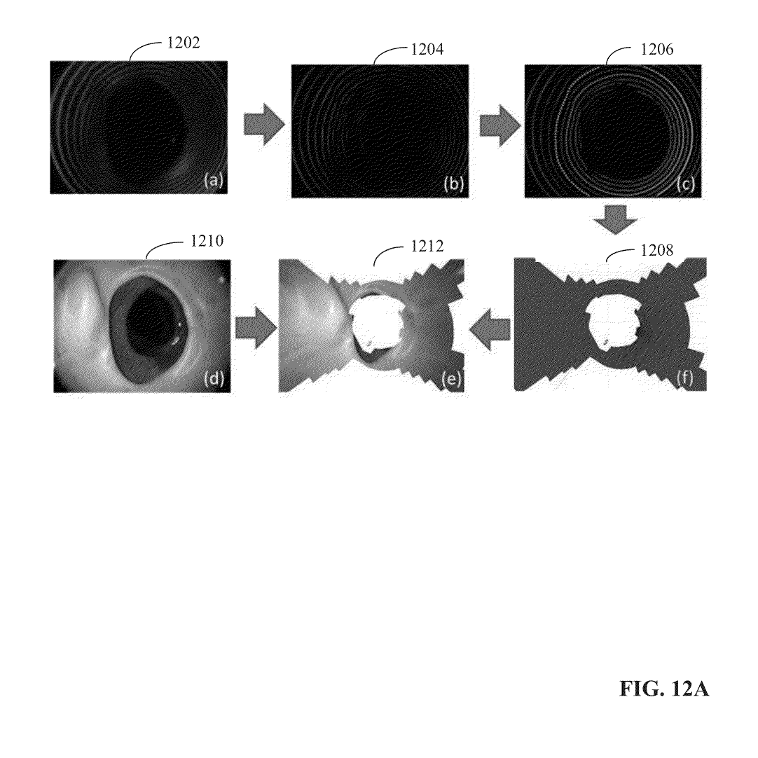

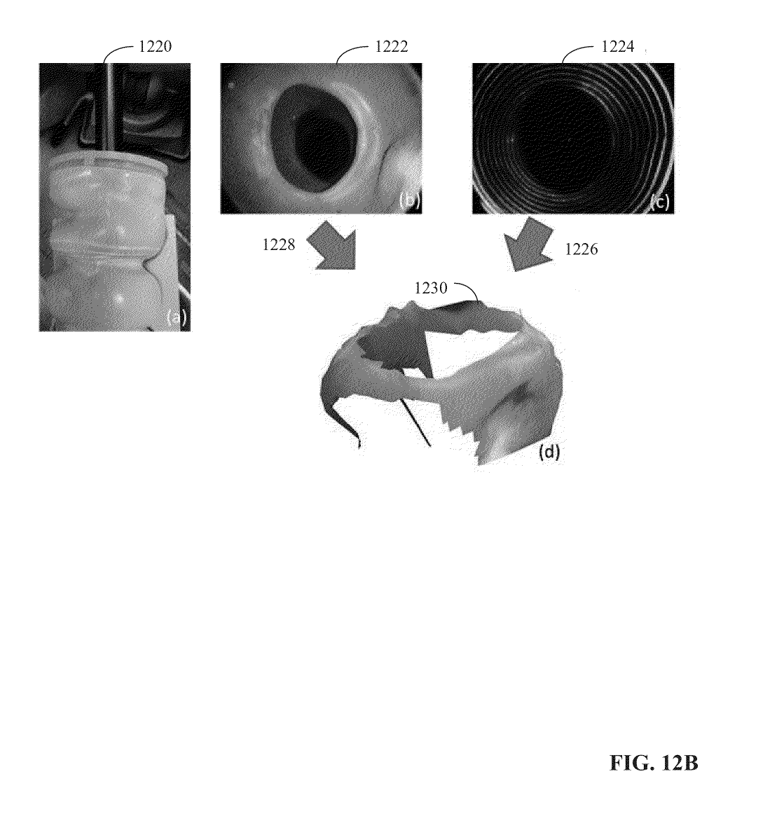

Reference is now made to FIG. 12A, which is a schematic flowchart graphically depicting the method of generating a colorized 3D reconstructed image and/or 3D model of the lumen based on registration of 3D reconstructed image(s) with acquired color, in accordance with some embodiments of the present invention. Reference will be made to the method of FIG. 2B.

Blocks 1202-1208 depict generation of the 3D reconstructed image(s). Images may be reconstructed based on uniformly projected rings (i.e., without angular coding), and/or reconstructed based on angular coding as described herein.

At 1202, an angular coded pattern of structured light is generated, for example, as described with reference to block 202. Alternatively (as shown for clarify), the pattern of light includes uniform rings (i.e., without angular coding).

At 1204, the received reflection of the projected light is filtered, for example, as described with reference to block 208.

At 1206, the light patterns are identified (denoted herein by different colors), for example, as described with reference to block 210.

At 1208, a 3D reconstructed image of the lumen wall is generated (for example, a triangulated 3D mesh) based on the identified light patterns, for example, as described with reference to block 212.

Block 1210 depicts an acquired image of the lumen based on projection of white light, for example, as described with reference to block 218.

At block 1212, the color data from the color image of block 1210 is registered with the triangulated 3D mesh to color the 3D mesh, for example, as described with reference to block 216.

Reference is now made to FIG. 12B, which is another schematic flowchart graphically depicting the method of generating a colorized 3D reconstructed image and/or 3D model of the lumen based on registration of 3D reconstructed image(s) with acquired color, in accordance with some embodiments of the present invention. Reference will be made to the method of FIG. 2B.

Block 1220 depicts a plastic model of the human colon used for testing the systems and/or methods described herein.

At 1222, the internal surface of the model is color imaged based on projection of white light within the lumen, for example, as described with reference to block 218.

At 1224, reflections of the angular coded light is received and filtered, for example, as described with reference to block 208. Alternatively (as shown for clarify), the pattern of received light includes uniform rings (i.e., without angular coding).

At 1226, the data based on the analyzed angular coding is used to generate a 3D reconstruction of the lumen, for example, as described with reference to block 212. Alternatively, the data based on the uniform rings is used to generate the 3D reconstruction.

At 1228, color data from image 1222 is registered with the 3D mesh, for example, as described with reference to block 216.

At 1230, the color 3D image and/or 3D model is provided, for example, as described with reference to block 214.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant systems and methods will be developed and the scope of the terms endoscope, sensor, projector and processor are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for generating a 3D reconstruction of an internal surface of a hollow lumen, comprising:
generating a light pattern having a code denoting angular positions;
projecting the light pattern onto an internal surface of a tubular lumen;
receiving reflections of the light pattern from the internal surface of the tubular lumen;
identifying angular positions of the light pattern based on the code; and
generating a 3D reconstruction of the internal surface from the received light pattern reflections based on the identified angular positions.

2. The method of claim 1, wherein the code denotes one or both of a position relative to an optical axis and an arc length.

3. The method of claim 1, wherein the light pattern has rotational symmetry.

4. The method of claim 1, wherein projecting comprises projecting the light pattern as a series of coaxial cones having different diverging angles, wherein a circumference of each coaxial cone is coded with the code denoting angular position.

5. The method of claim 1, wherein the code denoting angular position is selected to increase measurement precision at a direction perpendicular to a vector between a camera receiving reflections of the light pattern and a projector projecting the light patterns.

6. The method of claim 1, further comprising filtering the received light pattern reflection to resolve different parts of the projected light pattern.

7. The method of claim 6, wherein filtering comprises filtering to enhance pseudo-ellipsoidal rings within the received reflected light pattern.

8. The method of claim 6, wherein filtering comprises filtering to suppress enhancement of elongated patterns perpendicular to the direction of projected rings of the light pattern.

9. The method of claim 6, wherein filtering is based on the equation:
$${\hat{I}}_{c} ( x ; ξ ) = \{ \begin{matrix}0 , & if {λ}_{2} ( x ) > 0 \\ exp ( - \frac{{R}^{2} ( x )}{2 {σ}_{R}^{2}} ) ( 1 - exp ( - \frac{{S ( x )}^{2}}{2 {σ}_{S}^{2}} ) ) exp ( - \frac{{〈 {v}_{1} ( x ) , D ( x ) 〉}^{2}}{2 {σ}_{D}^{2}} ) , & otherwise\end{matrix}$$
wherein:
Î_{c} denotes a filtered image;
x denotes a spatial coordinate;
ξ denotes the Gaussian derivatives scale;
λ₁(x) and λ₂(x) denote the Hessian eigenvalues at location x, |λ₂(x)|≧|λ₁(x)|;
$$R ( x ) = \frac{{λ}_{1} ( x )}{{λ}_{2} ( x )} ,$$
the corresponding term promoting elongated structures;
S(x)=\sqrt{λ₁²+λ₂²}, is the Frobenius norm of the Hessian matrix, the corresponding term suppressing noise;
σ_{R}, σ_{S} are parameters controlling the filtering process;
v₁(x) is the Hessian eigenvector corresponding to λ₁, the eigenvalue with the lower magnitude;
$$D ( x ) = \frac{x - {x}_{0}}{‖ x - {x}_{0} ‖} ,$$
is the unit vector pointing from a projected cones axis x₀ to the pixel location x; and
σ_{D} is a parameter controlling a directional term.

10. The method of claim 1, further comprising:
modulating between projecting the light pattern and projecting multicolored light;
receiving reflections of the multicolored light; and
registering data based on the received reflection of the light pattern with data based on received reflection of the multicolored light to color the generated 3D reconstruction.

11. The method of claim 1, wherein the tubular lumen is selected from the group consisting of: trachea, bronchi, colon, esophagus, stomach, duodenum, bladder, fallopian tubes, uterus.

12. The method of claim 1, further comprising repeating the method to sequentially generate multiple 3D reconstructions, and registering the multiple reconstructions to generate a continuous 3D model of the internal surface.

13. A system for generating a 3D reconstruction of an internal surface of a hollow lumen, comprising:
a source of light for generating a light pattern having a code denoting angular positions, the source of light set for projection of the light pattern onto an internal surface of a tubular lumen;
a sensor for receiving reflections of the light pattern from the internal surface of the tubular lumen;
a processor in electrical communication with the sensor; and
a memory in electrical communication with the processor, the memory having stored thereon:
a module for identifying angular positions of the light pattern based on the code; and
a module for generating a 3D reconstruction of the internal surface from the received light pattern reflections based on the identified angular positions.

14. The system of claim 13, further comprising an endoscope sized for insertion into the tubular lumen, the sensor sized for insertion into the tubular lumen when coupled to a distal end region of the endoscope.

15. The system of claim 13, further comprising expanding optics in optical communication with a diffractive optical element of the source of light, the expanding optics arranged to project the light pattern across a wide field of view including the internal surface of the tubular lumen.

16. The system of claim 13, further comprising:
a colored illuminator for projecting colored light on the internal surface;
a module for modulating between projection of the light pattern and projection of the colored light; and
a module for registering received colored light with received reflection of the light pattern, and for coloring the generated 3D reconstruction based on the registration.

17. The system of claim 13, further comprising a curved mirror positioned distally to the source of light, the mirror sized for allowing some of the projected light pattern to fall on the internal surface distal to the sensor and for reflecting other portions of the projected light pattern to fall on the internal surface proximal to the sensor, the mirror designed based on the projected light pattern to maintain the integrity of the coding during simultaneous proximal and distal imaging.

18. The system of claim 17, wherein the mirror is configured to reflect the light reflected off the internal surface to the sensor.

19. The system of claim 17, wherein the mirror is configured such that both the projected light pattern and the light reflected back from the internal surface substantially maintain their respective integrity of the coding when reflected off the mirror.

20. A method for generating a 3D reconstruction of an internal surface of a hollow lumen, comprising:
receiving reflections from the internal surface of a tubular lumen, of a light pattern of projected cones having a code denoting angular positions;
filtering the received reflection to suppress enhancement of elongated patterns perpendicular to the direction of the projected cones;
identifying angular positions of the light pattern based on the code; and
generating a 3D reconstruction of the internal surface from the filtered light pattern reflections based on the identified angular positions.
